(19) United States
(12) Patent Application Publication
(10) Pub. No.: US 2025/0182904 A1
(43) Pub. Date: Jun. 5, 2025

(54) Analysis Module, Analysis System, Electronic Device And Analysis Method

(71) Applicant: Largan Health AI-Tech Co., Ltd., Taipei City (TW)

(72) Inventors: Cheng-Kuo LAI, Taipei City (TW); Yuan-Yen YAO, Taipei City (TW); Chen-Hsiang CHEN, Taipei City (TW); Yu Jie HONG, Taipei City (TW)

(21) Appl. No.: 18/951,804

(22) Filed: Nov. 19, 2024

(30) Foreign Application Priority Data
Dec. 1, 2023 (TW) 112146943

(51) Int. Cl. G16H 50/30

(52) U.S. Cl. CPC G16H 50/30

(57) ABSTRACT

An analysis module includes a user sleeping data extracting unit, a standard sleeping data extracting unit, a sleeping data analysis unit, a sleeping score conversion unit and a sleeping score displaying unit. The user sleeping data extracting unit is configured to extract a user sleeping data from an accessing module. The standard sleeping data extracting unit is configured to extract a standard sleeping data from the accessing module. The sleeping data analysis unit is configured to compare the user sleeping data with the standard sleeping data and generate sleeping analysis result. The sleeping score conversion unit is configured to convert the sleeping analysis result into a sleeping index grade, a sleeping quality grade and a sleeping index graphics. The sleeping score displaying unit is configured to display the sleeping index grade, the sleeping quality grade and the sleeping index graphics.

### RELATED APPLICATIONS

This application claims priority to Taiwan Application Serial Number 112146943, filed Dec. 1, 2023, which is herein incorporated by reference.

### BACKGROUND

### Technical Field

The present disclosure relates to an analysis module, an analysis system, an electronic device and an analysis method. More particularly, the present disclosure relates to an analysis module, an analysis system and an analysis method applicable to the electronic devices.

### Description of Related Art

Although existing wearable devices with sleeping monitoring functions can help users collect their sleeping data, the aforementioned wearable devices can only display sleeping data and cannot help users evaluate their own sleeping quality. Therefore, additional systems with analysis functions are needed. However, although the aforementioned system can help users analyze sleeping data, it provides sleeping analysis results in a complex presentation method, making it impossible for users to clearly understand their own sleeping quality. To help users quickly and accurately understand their own sleeping quality, it is necessary to actively develop methods that combine rapid analysis and accurate presentation.

### SUMMARY

According to one aspect of the present disclosure, an analysis module includes a user sleeping data extracting unit, a standard sleeping data extracting unit, a sleeping data analysis unit, a sleeping score conversion unit and a sleeping score displaying unit. The user sleeping data extracting unit is configured to extract a user sleeping data from an accessing module. The standard sleeping data extracting unit is configured to extract a standard sleeping data from the accessing module corresponding to the user sleeping data. The sleeping data analysis unit is configured to compare the user sleeping data with the standard sleeping data and generate at least one sleeping analysis result. The sleeping score conversion unit is configured to convert the at least one sleeping analysis result into at least one sleeping index grade, a sleeping quality grade and a sleeping index graphics. The sleeping score displaying unit is configured to display the at least one sleeping index grade, the sleeping quality grade and the sleeping index graphics, and the sleeping index graphics is a radar chart.

According to another aspect of the present disclosure, an analysis system includes the analysis module according to the aforementioned aspect and the accessing module connected to the analysis module.

According to another aspect of the present disclosure, an electronic device includes the analysis system according to the aforementioned aspect.

According to another aspect of the present disclosure, an analysis module includes a user sleeping data extracting unit, a standard sleeping data extracting unit, a sleeping data analysis unit, a sleeping score conversion unit and a sleeping score displaying unit. The user sleeping data extracting unit is configured to extract a user sleeping data from an accessing module. The standard sleeping data extracting unit is configured to extract a standard sleeping data from the accessing module corresponding to the user sleeping data. The sleeping data analysis unit is configured to compare the user sleeping data with the standard sleeping data and generate at least three sleeping analysis results. The sleeping score conversion unit is configured to convert the at least three sleeping analysis results into at least three sleeping index grades, at least one sleeping quality grade and a sleeping index graphics, and the at least three sleeping analysis results includes at least one of slow wave power, breathing power, falling asleep power, sustainability and sleeping length power. The sleeping score displaying unit is configured to display the at least three sleeping index grades, the sleeping quality grade and the sleeping index graphics.

According to another aspect of the present disclosure, an analysis system includes the analysis module according to the aforementioned aspect and the accessing module connected to the analysis module.

According to another aspect of the present disclosure, an electronic device includes the analysis system according to the aforementioned aspect.

According to another aspect of the present disclosure, an analysis method includes the following steps. An analysis module is driven to extract a user sleeping data from an accessing module. The analysis module is driven to extract a standard sleeping data from the accessing module corresponding to the user sleeping data. The analysis module is driven to compare the user sleeping data with the standard sleeping data and generate at least one sleeping analysis result. The analysis module is driven to convert the at least one sleeping analysis result into at least one sleeping index grade, a sleeping quality grade and a sleeping index graphics. The analysis module is driven to display the at least one sleeping index grade, the sleeping quality grade and the sleeping index graphics, and the sleeping index graphics is a radar chart.

### BRIEF DESCRIPTION OF THE DRAWINGS

The present disclosure can be more fully understood by reading the following detailed description of the embodiment, with reference made to the accompanying drawings as follows:

FIG. 1 is a block diagram of an analysis system according to the 1st embodiment of the present disclosure.

FIG. 2 is a block diagram of an analysis module according to the 1st embodiment of FIG. 1.

FIG. 3 is a schematic view of some units of the analysis module respectively displayed on a user interface according to the 1st embodiment of FIG. 2.

FIG. 4 is a schematic view of the user interface according to the 1st example of the 1st embodiment of FIG. 2.

FIG. 5 is a schematic view of the user interface according to the 2nd example of the 1st embodiment of FIG. 2.

FIG. 6 is a schematic view of a sleeping index graphics according to the 3rd example of the 1st embodiment of FIG. 2.

FIG. 7 is a schematic view of a sleeping index graphics according to the 4th example of the 1st embodiment of FIG. 2.

FIG. 8 is a schematic view of a sleeping index graphics according to the 5th example of the 1st embodiment of FIG. 2.

FIG. 9 is a flow chart of an analysis method according to the 2nd embodiment of the present disclosure.

FIG. 10A is a schematic view of an electronic device according to the 3rd embodiment of the present disclosure.

FIG. 10B is a block diagram of the electronic device according to the 3rd embodiment of FIG. 10A.

### DETAILED DESCRIPTION

An analysis module includes an analysis module includes a user sleeping data extracting unit, a standard sleeping data extracting unit, a sleeping data analysis unit, a sleeping score conversion unit and a sleeping score displaying unit. The user sleeping data extracting unit is configured to extract a user sleeping data from an accessing module. The standard sleeping data extracting unit is configured to extract a standard sleeping data from the accessing module corresponding to the user sleeping data. The sleeping data analysis unit is configured to compare the user sleeping data with the standard sleeping data and generate at least one sleeping analysis result. The sleeping score conversion unit is configured to convert the at least one sleeping analysis result into at least one sleeping index grade, a sleeping quality grade and a sleeping index graphics. The sleeping score displaying unit is configured to display the at least one sleeping index grade, the sleeping quality grade and the sleeping index graphics, and the sleeping index graphics is a radar chart. The analysis module converts complex sleeping data into simple sleeping analysis result through the comparison and analysis functions of the analysis module so as to greatly improve analysis efficiency. The analysis module provides users with easy-to-understand sleeping quality analysis through a variety of display functions. Therefore, it is favorable for helping users to improve their understanding of their own sleeping quality. By displaying the radar chart, it is favorable for presenting the distribution of the pros and cons of each sleeping index grade concisely so as to evaluate and improve the sleeping analysis result.

The sleeping analysis result includes at least one of slow wave power, breathing power, falling asleep power, sustainability and sleeping length power. By analyzing the slow wave power, breathing power, falling asleep power, sustainability and sleeping length power which are the most related to sleeping quality, it is favorable for providing the most accurate sleeping quality analysis so as to improve the sleeping assessment accuracy of the analysis module.

The sleeping index grade is arranged around the sleeping index graphics. By displaying sleeping index grade and sleeping index graphics at the same time, there is no requirement for users to review data and graphics on multiple screens. Therefore, it is favorable for improving reading convenience.

The sleeping score displaying unit displays an indicator numeric score. By scoring in numeric, it is favorable for providing more intuitive scoring method for users who are accustomed to comparing numbers.

The sleeping score displaying unit displays a full score of indicator numeric, and the full score of indicator numeric overlaps the sleeping index graphics. By displaying the full score of indicator numeric and sleeping index graphics at the same time, it is favorable for quickly understanding the pros and cons of various sleeping analysis results on the sleeping index graphics.

The sleeping score displaying unit displays a quality letter grade. By scoring in English letters, it is favorable for providing more intuitive scoring method for users who are accustomed to comparing English letters.

The sleeping score displaying unit displays a full score of quality numeric. By displaying the sleeping quality grade and the full score of quality numeric at the same time, it is favorable for quickly understanding the pros and cons of sleeping quality.

The sleeping score displaying unit displays an indicator percentage. By displaying the indicator percentage, it is favorable for understanding the pros and cons of sleeping indicators among data groups.

The sleeping score displaying unit displays a quality percentage. By displaying the quality percentage, it is favorable for understanding the pros and cons of sleeping quality among data groups.

The analysis module can further include a sleeping interval displaying unit. Therefore, users can quickly check their sleeping intervals by the sleeping interval displaying unit, so that it is favorable for improving system convenience.

The analysis module can further include a sleeping position displaying unit. Therefore, it can provide sleeping position data during sleeping by the sleeping position displaying unit, so that it is favorable for understanding their sleeping position habits.

The sleeping index graphics is a donut chart, the donut chart is transformed by at least two sleeping analysis results, and the at least two sleeping analysis results include sleeping position and sleeping breathing. By displaying sleeping position and sleeping breathing in the donut chart at the same time, it is favorable for understanding the correlation between sleeping position and sleeping breathing so as to find the best sleeping position to improve sleeping quality.

The sleeping index graphics is a curve chart, the curve chart is transformed by at least one sleeping analysis result, and the at least one sleeping analysis result includes a number of times of rolling over and a number of times of getting out from a bed. By displaying the number of times of rolling over in the curve chart, it is favorable for understanding the suitability of bedding and improving sleeping quality by selecting bedding. Further, by displaying the number of times of getting out from the bed in the curve chart, it is favorable for understanding the degree of frequent urination.

The analysis module can further include a sleeping advice displaying unit. Therefore, users can be given directions to improve their sleeping quality by the sleeping suggestion function of the sleeping advice displaying unit, so that it is favorable for users upgrading their sleeping quality.

Each of the aforementioned technical features of the analysis module can be combined with each other and reach the corresponded effect.

The analysis module of the present disclosure refers to a processor, a microprocessor, a central processing unit (CPU), a mobile device processor, a cloud processor or other electronic computing processors with the functions of data extraction, comparison and analysis.

The sleeping data of the present disclosure refers to measurable data related to sleep. The sleeping data includes deep sleeping duration, duration of sleep, duration in bed, number of times of respiratory event, time of first light sleep, number of times of getting out from the bed, blood oxygen concentration, sleeping position, sleeping position time, number of times of rolling over, heartbeat, blood pressure, ECG rhythm, etc. The analysis module can extract at least one sleeping data, at least two sleeping data, at least three sleeping data, at least four sleeping data, at least five sleeping data, at least six sleeping data, and at least seven sleeping data, at least eight sleeping data, at least nine sleeping data, and at least ten sleeping data.

The user sleeping data extracting unit of the present disclosure can extract at least one user sleeping data, at least two user sleeping data, at least three user sleeping data, at least four user sleeping data, at least five user sleeping data, at least six user sleeping data, at least seven user sleeping data, at least eight user sleeping data, at least nine user sleeping data, or at least ten user sleeping data from the accessing module. The user sleeping data refers to the sleeping data of a specific user, which is usually obtained through measurements by sleeping monitoring equipment.

The standard sleeping data extracting unit of the present disclosure can extract at least one standard sleeping data, at least two standard sleeping data, at least three standard sleeping data, at least four standard sleeping data, at least five standard sleeping data, at least six standard sleeping data, at least seven standard sleeping data, at least eight standard sleeping data, at least nine standard sleeping data, or at least ten standard sleeping data from the accessing module. The standard sleeping data refers to sleeping data used as a comparison standard. The standard sleeping data is usually taken from research results. The standard sleeping data will change depending on the user's age, gender, etc. For example: if the user is a 35-year-old male, the standard sleeping data can be the optimal sleeping data for men aged 30 to 39 provided by a sleeping research institution; if the user is a 40-year-old female, the standard sleeping data can be the optimal sleeping data for women aged 40 to 49 provided by the sleeping research institution; if the user is an 11-year-old male, the standard sleeping data can be the optimal sleeping data for men aged 7 to 11 provided by the sleeping research institution; if the user is an 18-year-old female, the standard sleeping data can be the optimal sleeping data for women aged 12 to 18 provided by the sleeping research institution.

The sleeping data analysis unit of the present disclosure can compare the user sleeping data with the standard sleeping data and generate the sleeping analysis result.

The sleeping analysis result of the present disclosure is the result of comparing the user sleeping data with standard sleeping data and analyzing it. The sleeping analysis result can also be replaced by the term sleeping ability. Each sleeping analysis result can be selected from slow wave power, breathing power, falling asleep power, sustainability and sleeping length power. Each sleeping analysis result can also be selected from deep sleeping duration, duration of sleep, duration in bed, number of times of respiratory event, time of first light sleep, number of times of getting out from the bed, blood oxygen concentration, sleeping position, sleeping position time, number of times of rolling over, heartbeat, blood pressure, ECG rhythm, etc.

The slow wave power of the present disclosure is obtained by calculating the proportion of deep sleeping duration to sleeping duration, and is a sleeping indicator that evaluates the quality of deep sleep. The calculation formula of slow wave power is as follow: deep sleeping duration/sleeping duration; wherein the deep sleeping duration is the total duration of the deep sleeping period marked by the ECG signal. The slow wave force can also be replaced by terms such as slow wave proportion and slow wave sleep.

The breathing power of the present disclosure is obtained by calculating the number of times of respiratory event in sleeping duration, and is a sleeping indicator that evaluates the respiratory event disorders. The calculation formula of breathing power is as follow: the number of times of respiratory event/sleeping duration; wherein the respiratory event refers to abnormal breathing, such as respiratory cessation, changes in respiratory frequency, etc. The breathing power can also be replaced by terms such as sleeping breathing and breathing sleeping.

The falling asleep power of the present disclosure is obtained by calculating the time to first fall into light sleep, and is a sleeping indicator that evaluates sleeping disorder; wherein the falling asleep power refers to how long it takes to fall asleep after going to bed. The falling asleep power can also be replaced by terms such as ability of fall asleep and speed of falling asleep.

The sustainability of the present disclosure is obtained by calculating the proportion of sleeping duration to duration in bed and subtracting the number of times of getting out from the bed, and is a sleeping indicator that evaluates the problem of easy awakening. The calculation formula of sustainability is as follow: (sleeping duration/duration in bed)−number of times of getting out from the bed; wherein the duration in bed is the total time from the user lies in bed to gets out of bed. The sustainability can also be replaced by terms such as sleeping persistence and sustained sleep.

The sleeping length power of the present disclosure is obtained by calculating the time of duration in bed, and is a sleeping indicator that evaluates whether sleep is adequate. The sleeping length power can also be replaced by terms such as sleeping length and total sleeping length.

The sleeping score conversion unit of the present disclosure can convert the sleeping analysis result into the sleeping index grade, sleeping quality grade and sleeping index graphics.

The sleeping score displaying unit of the present disclosure can display the sleeping index grade, the sleeping quality grade and the sleeping index graphics. The sleeping index grade and the sleeping quality grade can be selected arbitrarily and arranged around or overlapping the sleeping index graphics.

The sleeping index grade of the present disclosure refers to the score converted from the sleeping analysis result, which can represent the pros and cons of each sleeping analysis result. The sleeping index grade can be converted from slow wave power, breathing power, falling asleep power, sustainability and sleeping length power. The sleeping index grade can include indicator numeric score, full score of indicator numeric, indicator letter grade, indicator text grade and indicator percentage.

The indicator numeric score of the present disclosure can be an integer number or a non-integer number between 0˜1, 0˜10, 0˜100, 0˜500 or 0˜1000, and the number is used as the criterion for excellence. The upper limit of indicator numeric score is the full score of indicator numeric. The full score of indicator numeric can be 1, 5, 10, 20, 30, 50, 100, 200, 500 or 1000. When the scoring method of the sleeping index grade is numeric, the indicator numeric score and the full score of indicator numeric can be displayed at the same time. For example: the indicator numeric score and the full score of indicator numeric can be displayed as 0.1/1, 4.1/5, 8.9/10 and 11.1/20.

The indicator letter grade of the present disclosure can includes A, B, C, D, E, F, G, H, I, J, K, L, M, N, O, P, Q, R, S, T, U, V, W, X, Y or Z, and can customize the pros and cons represented by the English letters. For example: the full score of indicator letter grade can be A, the full score of indicator letter grade can be A+, the full score of indicator letter grade can be S, the full score of indicator letter grade can be S+, the full score of indicator letter grade can be SS, the full score of indicator letter grade can be SSS, and the full score of indicator letter grade can be L.

The indicator text grade of the present disclosure can includes terms such as outstanding, excellent, good, fair, poor or bad, and score the full score after customizing the text combination. For example: the full score of indicator text grade can be outstanding, excellent, very good or good.

The indicator percentage of the present disclosure is obtained by comparing the user's sleeping analysis result with the data group's sleeping analysis results, and refers to the proportion of pros and cons of a single sleeping analysis result in the data group. For example: the indicator percentage is 80%, which means that the user's sleeping analysis result is better than 80% of other users in the data group. The data group of the indicator percentage can be filtered based on age, gender, occupation, race, region and country, etc. The indicator percentage can be obtained by comparing slow wave power, breathing power, falling asleep power, sustainability, sleeping length power and other analysis results.

The sleeping quality grade of the present disclosure is refers to a score converted from a plurality of sleeping analysis results, which can represent the comprehensive pros and cons of multiple sleeping analysis results. The sleeping quality grade can be converted from at least one sleeping analysis result, at least two sleeping analysis results, at least three sleeping analysis results, at least four sleeping analysis results, at least five sleeping analysis results, at least six sleeping analysis results, at least seven sleeping analysis results, at least eight sleeping analysis results, at least nine sleeping analysis results, or at least ten sleeping analysis results. The plurality of sleeping analysis results converted into the sleeping quality grade can be selected from at least one of slow wave power, breathing power, falling asleep power, sustainability and sleeping length power. The sleeping quality grade can include quality numeric score, full score of quality numeric, quality letter grade, quality text grade and quality percentage.

The quality numeric score of the present disclosure can be an integer number or a non-integer number between 0˜1, 0˜10, 0˜100, 0˜500 or 0˜1000, and the number is used as the criterion for excellence. The upper limit of quality numeric score is the full score of quality numeric. The full score of quality numeric can be 1, 5, 10, 20, 30, 50, 100, 200, 500 or 1000. When the scoring method of the sleeping quality grade is numeric, the quality numeric score and the full score of quality numeric can be displayed at the same time. For example: the quality numeric score and the full score of quality numeric can be displayed as 0.1/1, 4.1/5, 8.9/10 and 11.1/20.

The quality letter grade of the present disclosure can includes A, B, C, D, E, F, G, H, I, J, K, L, M, N, O, P, Q, R, S, T, U, V, W, X, Y or Z, and can customize the pros and cons represented by the English letters. For example: the full score of quality letter grade can be A, the full score of quality letter grade can be A+, the full score of quality letter grade can be S, the full score of quality letter grade can be S+, the full score of quality letter grade can be SS, the full score of quality letter grade can be SSS, and the full score of quality letter grade can be L.

The quality text grade of the present disclosure can includes terms such as outstanding, excellent, good, fair, poor or bad, and score the full score after customizing the text combination. For example: the full score of quality text grade can be outstanding, excellent, very good or good.

The quality percentage of the present disclosure is obtained by comparing multiple user's sleeping analysis results with the data group's sleeping analysis results, and refers to the proportion of pros and cons of sleeping quality in the data group. For example: the quality percentage is 80%, which means that the user's sleeping quality is better than 80% of other users in the data group. The data group of the quality percentage can be filtered based on age, gender, occupation, race, region and country, etc. The quality percentage can be obtained by comparing at least one sleeping analysis result, at least two sleeping analysis results, at least three sleeping analysis results, at least four sleeping analysis results, at least five sleeping analysis results, at least six sleeping analysis results, at least seven sleeping analysis results, at least eight sleeping analysis results, at least nine sleeping analysis results, or at least ten sleeping analysis results. The plurality of sleeping analysis results compared to the sleeping quality grade can be selected from at least one, at least two, at least three or at least four of slow wave power, breathing power, falling asleep power, sustainability and sleeping length power.

The sleeping index graphics of the present disclosure refer to graphics transformed from sleeping analysis results, and can indicate the pros and cons of various sleeping analysis results. The sleeping index graphics can be radar chart, donut chart, bell curves, curve chart, circle chart, pie chart, bar chart, horizontal bar chart, line chart, and X-Y scatters graph, surface plot, or radial donut plot. The sleeping index graphics can include at least one sleeping analysis result, at least two sleeping analysis results, at least three sleeping analysis results, at least four sleeping analysis results, at least five sleeping analysis results, at least six sleeping analysis results, at least seven sleeping analysis results, at least eight sleeping analysis results, at least nine sleeping analysis results, or at least ten sleeping analysis results. The sleeping analysis results included in the sleeping index graphics can be selected from at least one, at least two, at least three or at least four of slow wave power, breathing power, falling asleep power, sustainability and sleeping length power.

The sleeping interval displaying unit of the present disclosure is configured to display the user's sleeping interval. The sleeping interval refers to the time interval from the first non-awake time to the last non-awake time. If the user has insomnia, the sleeping interval will be based on the last non-awake time that the user stops trying to sleep clearly. The sleeping interval is usually measured by sleeping monitoring equipment.

The sleeping position displaying unit is configured to display the user's sleeping position time. The sleeping position time refers to the total time of the user spends in various sleeping positions during the sleeping interval. The sleeping positions can include supine position, left lateral position, right lateral position, prone position and other sleeping positions. The sleeping position time is usually measured by sleeping monitoring equipment.

The sleeping advice displaying unit provides sleeping advice based on a sleeping analysis result and displays the sleeping advice. The sleeping advice refers to suggestions that favorable for users improving their sleeping quality.

An analysis system includes the aforementioned analysis module and the aforementioned accessing module, wherein the analysis module can extract the sleeping data from accessing module, and compare and analyze it.

The accessing module of the present disclosure is composed of a database. The database can store a large amount of sleeping data. The database can be a local database or a network database. In detail, the database can be memory, disk drive or hard disk. The accessing module can includes at least one database, at least two databases, at least three databases, at least four databases, at least five databases, at least six databases, at least seven databases, at least eight databases, at least nine databases, or at least ten databases. In addition, the accessing module can include at most thousands to tens of thousands of databases, wherein the databases can be the first database, the second database, the third database, the fourth database, the fifth database, the sixth database, the seventh database, the eighth database, the ninth database, the tenth database, and so on. The memory can be a random access memory (RAM) or other types of dynamic storage devices that can store information and instructions for execution by the analysis module.

An electronic device includes the aforementioned analysis system and a user interface. The electronic device of the present disclosure can be mobile phones, sleeping monitoring instruments, tablet computers, desktop computers, notebook, smart bracelets, smart watches and electronic wearable devices. The user interface of the present disclosure can be a touch screen or a display screen.

An analysis method includes driving the aforementioned analysis module to extract the user sleeping data from the aforementioned accessing module, driving the analysis module to extract the standard sleeping data from the accessing module corresponding to the user sleeping data, driving the analysis module to compare the user sleeping data with the standard sleeping data and generate sleeping analysis result, driving the analysis module to convert the sleeping analysis result into the sleeping index grade, the sleeping quality grade and the sleeping index graphics, driving the analysis module to display the sleeping index grade, the sleeping quality grade and the sleeping index graphics, and the sleeping index graphics is a radar chart. Therefore, it is favorable for greatly improving the analysis efficiency by the method that can quantify various sleep, and it is favorable for providing easy-to-understand sleeping quality analysis by the method that can easily present sleeping quality so as to help users to improve their understanding of their own sleeping quality. By displaying the radar chart, the distribution of the pros and cons of each sleeping index grade is presented concisely, which helps users evaluate and improve the sleeping analysis results.

According to the above embodiment, specific examples are proposed below and explained in detail with the drawings.

### 1st Embodiment

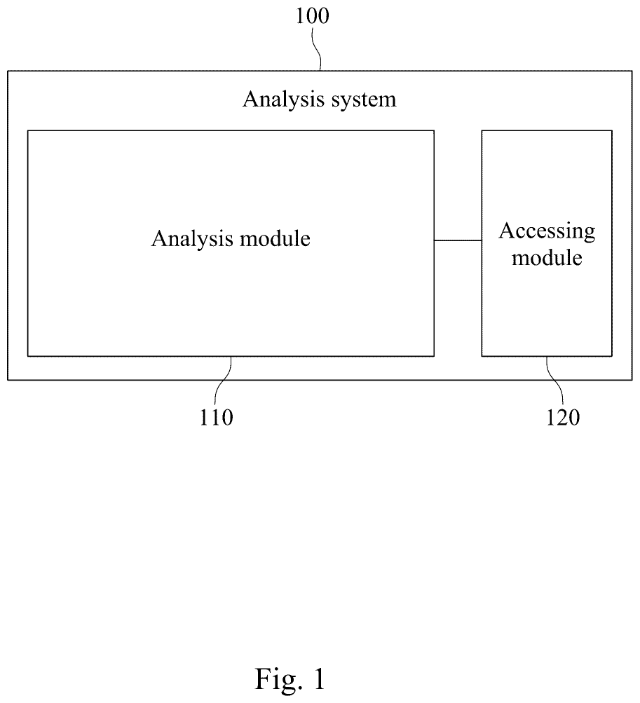

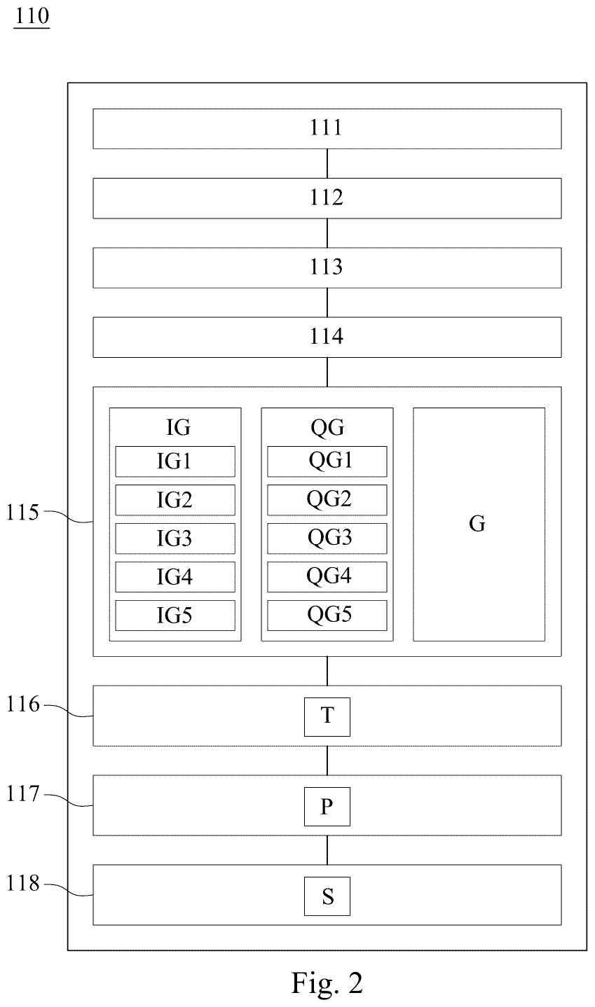

FIG. 1 is a block diagram of an analysis system 100 according to the 1st embodiment of the present disclosure. FIG. 2 is a block diagram of an analysis module 110 according to the 1st embodiment of FIG. 1. In the 1st embodiment, the analysis system 100 can be applied to an electronic device such as a computer or a mobile phone (not shown), and the analysis system 100 can be signally connected to a sleeping monitoring instrument (not shown), or the analysis system 100 can be applied in the sleeping monitoring instrument to obtain various physiological activity indices of the human body during sleep (such as heartbeat, body temperature, blood pressure, sleeping time, sleeping position and respiratory rate) for analysis.

The analysis system 100 includes an analysis module 110 and an accessing module 120, the analysis module 110 is signally connected to the accessing module 120. The analysis module includes a user sleeping data extracting unit 111, a standard sleeping data extracting unit 112, a sleeping data analysis unit 113, a sleeping score conversion unit 114, a sleeping score displaying unit 115, a sleeping interval displaying unit 116, a sleeping position displaying unit 117 and a sleeping advice displaying unit 118. The user sleeping data extracting unit 111 is configured to extract a user sleeping data from the accessing module 120. The standard sleeping data extracting unit 112 is configured to extract a standard sleeping data from the accessing module 120 corresponding to the user sleeping data. In the 1st embodiment, the user sleeping data refers to the sleeping data of a specific user, which is obtained through measurements by sleeping monitoring equipment; the standard sleeping data refers to sleeping data used as a comparison standard, and the standard sleeping data is taken from research results.

The sleeping data analysis unit 113 is configured to compare the user sleeping data with the standard sleeping data and generate at least one sleeping analysis result. The sleeping score conversion unit 114 is configured to convert the sleeping analysis results into at least one sleeping index grade IG, a sleeping quality grade QG and a sleeping index graphics G. In the 1st embodiment, the sleeping analysis result includes at least one of slow wave power, breathing power, falling asleep power, sustainability and sleeping length power. The sleeping index grade IG refers to the score converted from the sleeping analysis result, which can represent the pros and cons of each sleeping analysis result. The sleeping quality grade QG refers to a score converted from a plurality of sleeping analysis results, which can represent the comprehensive pros and cons of multiple sleeping analysis results. The sleeping index graphics G refer to graphics transformed from sleeping analysis results, the sleeping index graphics G can indicate the pros and cons of various sleeping analysis results.

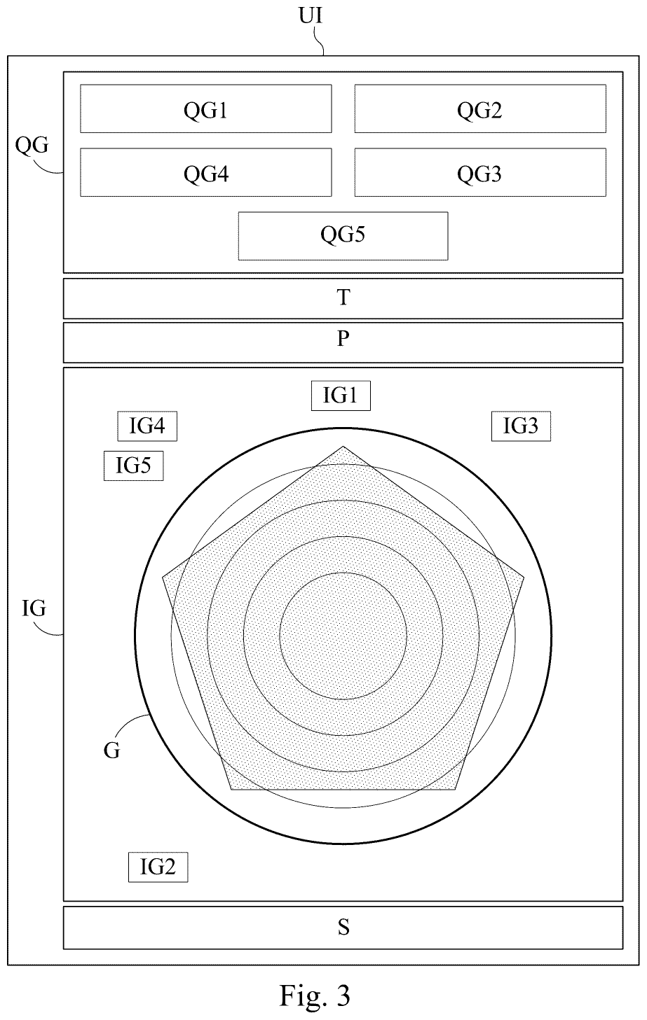

FIG. 3 is a schematic view of some units of the analysis module 110 respectively displayed on a user interface UI according to the 1st embodiment of FIG. 2. In FIG. 2 and FIG. 3, the sleeping score displaying unit 115 is configured to display the sleeping index grades IG, the sleeping quality grade QG and the sleeping index graphics G. The sleeping interval displaying unit 116 is configured to display the user's sleeping interval T. The sleeping position displaying unit 117 is configured to display the user's sleeping position data P during sleep. The sleeping advice displaying unit 118 is configured to provide sleeping advice S based on sleeping analysis result and displays the sleeping advice S. In the 1st embodiment, the sleeping index grade IG includes an indicator numeric score IG1, a full score of indicator numeric IG2, an indicator letter grade IG3, a text grade IG4 and an indicator percentage IG5. The sleeping quality grade QG includes a quality numeric score QG1, a full score of quality numeric QG2, a quality letter grade QG3, a quality text grade QG4 and a quality percentage QG5. The sleeping index graphics G is a radar chart. The sleeping score displaying unit 115 can display the indicator numeric score IG1, the full score of indicator numeric IG2, the indicator letter grade IG3, the text grade IG4, the indicator percentage IG5, the quality numeric score QG1, the full score of quality numeric QG2, the quality letter grade QG3, the quality text grade QG4, the quality percentage QG5 and the sleeping index graphics G on the user interface UI of the electronic device (not shown) or the sleeping monitoring instrument (not shown). The sleeping interval displaying unit 116 can display the sleeping interval T on the user interface UI of the electronic device (not shown) or the sleeping monitoring instrument (not shown). The sleeping position displaying unit 117 can display the sleeping position data P on the user interface UI of the electronic device (not shown) or the sleeping monitoring instrument (not shown). The sleeping advice displaying unit 118 can display the sleeping advice S on the user interface UI of the electronic device (not shown) or the sleeping monitoring instrument (not shown). It should be noted that the sleeping interval T, the sleeping position data P and the sleeping advice S can be displayed or not displayed by the user at will.

In the 1st embodiment, the sleeping quality grade QG is displayed at the top of the user interface UI, the sleeping advice S is displayed at the bottom of the user interface UI, the sleeping interval T, the sleeping position data P and the sleeping index graphics G are displayed sequentially from top to bottom between the sleeping quality grade QG and the sleeping advice S. The sleeping index grade IG is distributed and arranged around the sleeping index graphics G. In detail, the indicator numeric score IG1, the full score of indicator numeric IG2, the indicator letter grade IG3, the text grade IG4 and the indicator percentage IG5 of the sleeping index grade IG are distributed and arranged around the sleeping index graphics G. In other examples, the distribution of the indicator numeric score IG1, the full score of indicator numeric IG2, the indicator letter grade IG3, the text grade IG4 and the indicator percentage IG5 can be selected arbitrarily, and can be arbitrarily arranged around or overlapped with the sleeping index graphics G.

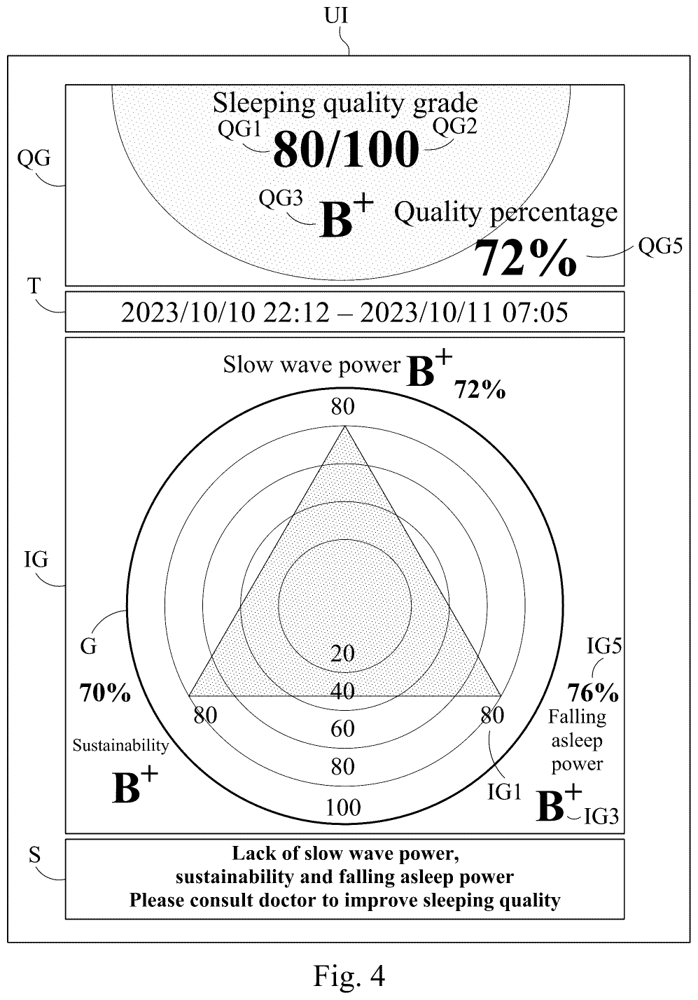

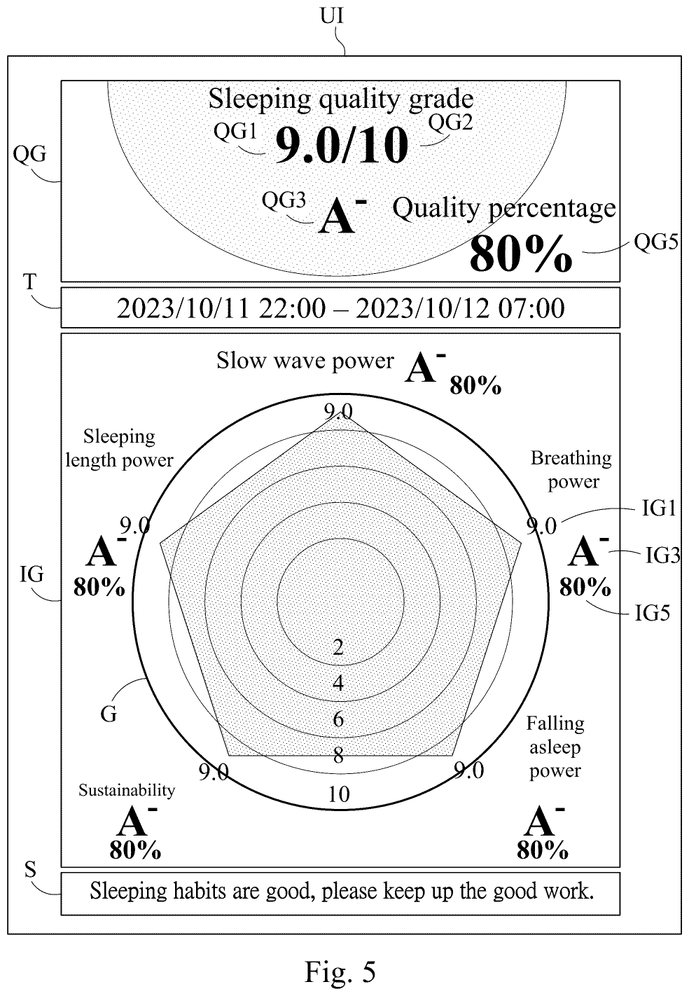

FIG. 4 is a schematic view of the user interface UI according to the 1st example of the 1st embodiment of FIG. 2. FIG. 5 is a schematic view of the user interface UI according to the 2nd example of the 1st embodiment of FIG. 2.

According to FIG. 2, FIG. 4 and FIG. 5, in the 1st example of the 1st embodiment, the sleeping data analysis unit 113 compares the user sleeping data with the standard sleeping data and generate sleeping analysis results corresponding to slow wave power, falling asleep power and sustainability. The sleeping score conversion unit 114 converts the sleeping analysis results corresponding to slow wave power, falling asleep power and sustainability into sleeping quality grade QG, sleeping index grade IG and sleeping index graphics G. As shown in FIG. 4, the sleeping score displaying unit 115 displays the sleeping quality grade QG, sleeping index grade IG and sleeping index graphics G on the user interface UI. The sleeping interval displaying unit 116 displays the sleeping interval T on the user interface UI. The sleeping advice displaying unit 118 displays the sleeping advice S on the user interface UI. The sleeping quality grade QG displayed by the sleeping score displaying unit 115 is the quality numeric score QG1, the full score of quality numeric QG2, the quality letter grade QG3 and the quality percentage QG5. The sleeping index grade IG displayed by the sleeping score displaying unit 115 is the indicator numeric score IG1, the indicator letter grade IG3 and the indicator percentage IG5. The sleeping index graphics G displayed by the sleeping score displaying unit 115 is radar chart corresponding to slow wave power, falling asleep power and sustainability, wherein, the indicator numeric score IG1 is overlapped with the sleeping index graphics G. The quality numeric score QG1 and the indicator numeric score IG1 are integer number or non-integer number between 0˜100, and the number is used as the criterion for excellence, and the full score of quality numeric QG2 is 100. The quality letter grade QG3 and the indicator letter grade IG3 are English letters such as A+, A, A−, B+, B, B−, etc., A+ corresponds to the quality numeric score QG1 and the indicator numeric score IG1 between greater than 95 and less than or equal to 100, A corresponds to the quality numeric score QG1 and the indicator numeric score IG1 between greater than 90 and less than or equal to 95, A-corresponds to the quality numeric score QG1 and the indicator numeric score IG1 between greater than 85 and less than or equal to 90, B+ corresponds to the quality numeric score QG1 and the indicator numeric score IG1 between greater than 80 and less than or equal to 85, and so on.

In the 2nd example of the 1st embodiment, the sleeping data analysis unit 113 compares the user sleeping data with the standard sleeping data and generate sleeping analysis results corresponding to slow wave power, breathing power, falling asleep power, sustainability and sleeping length power. The sleeping score conversion unit 114 converts the sleeping analysis results corresponding to slow wave power, breathing power, falling asleep power, sustainability and sleeping length power into sleeping quality grade QG, sleeping index grade IG and sleeping index graphics G. As shown in FIG. 5, the sleeping score displaying unit 115 displays the sleeping quality grade QG, sleeping index grade IG and sleeping index graphics G on the user interface UI. The sleeping interval displaying unit 116 displays the sleeping interval T on the user interface UI. The sleeping advice displaying unit 118 displays the sleeping advice S on the user interface UI. The sleeping quality grade QG displayed by the sleeping score displaying unit 115 is the quality numeric score QG1, the full score of quality numeric QG2, the quality letter grade QG3 and the quality percentage QG5. The sleeping index grade IG displayed by the sleeping score displaying unit 115 is the indicator numeric score IG1, the indicator letter grade IG3 and the indicator percentage IG5. The sleeping index graphics G displayed by the sleeping score displaying unit 115 is radar chart corresponding to slow wave power, breathing power, falling asleep power, sustainability and sleeping length power, wherein, the indicator numeric score IG1 is overlapped with the sleeping index graphics G. The quality numeric score QG1 and the indicator numeric score IG1 are integer number or non-integer number between 0˜10, and the number is used as the criterion for excellence, and the full score of quality numeric QG2 is 10. The quality letter grade QG3 and the indicator letter grade IG3 are English letters such as A+, A, A−, B+, B, B−, etc., A+ corresponds to the quality numeric score QG1 and the indicator numeric score IG1 between greater than 9.5 and less than or equal to 10, A corresponds to the quality numeric score QG1 and the indicator numeric score IG1 between greater than 9.0 and less than or equal to 9.5, A− corresponds to the quality numeric score QG1 and the indicator numeric score IG1 between greater than 8.5 and less than or equal to 9.0, and so on.

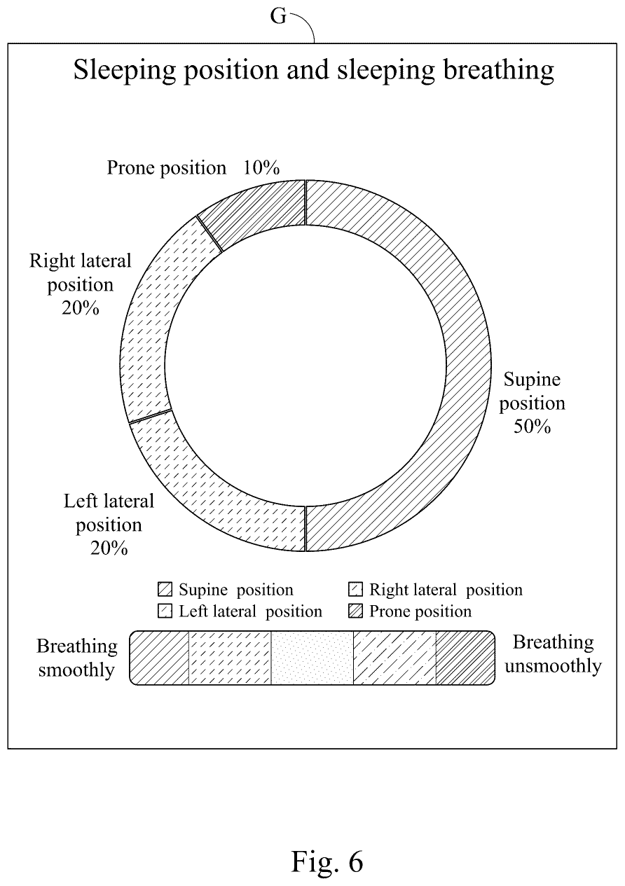

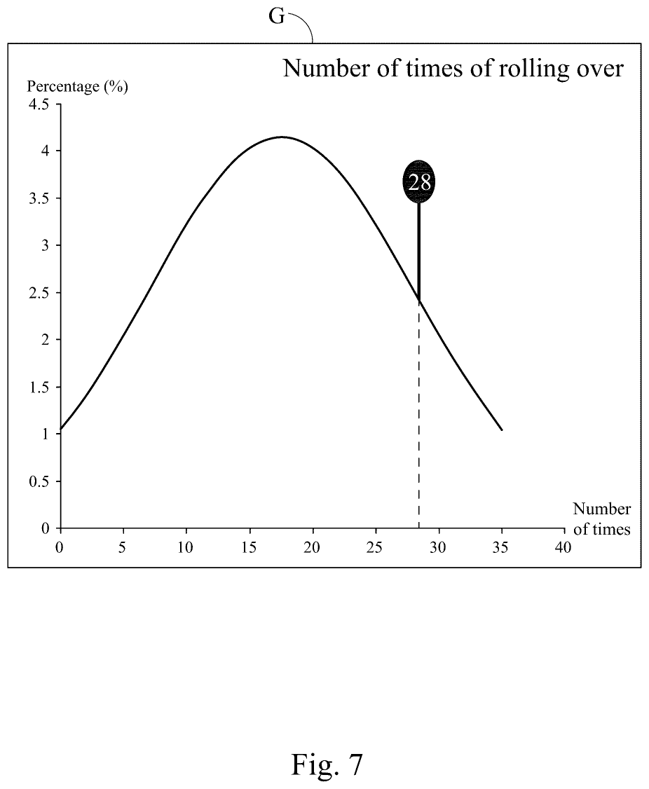

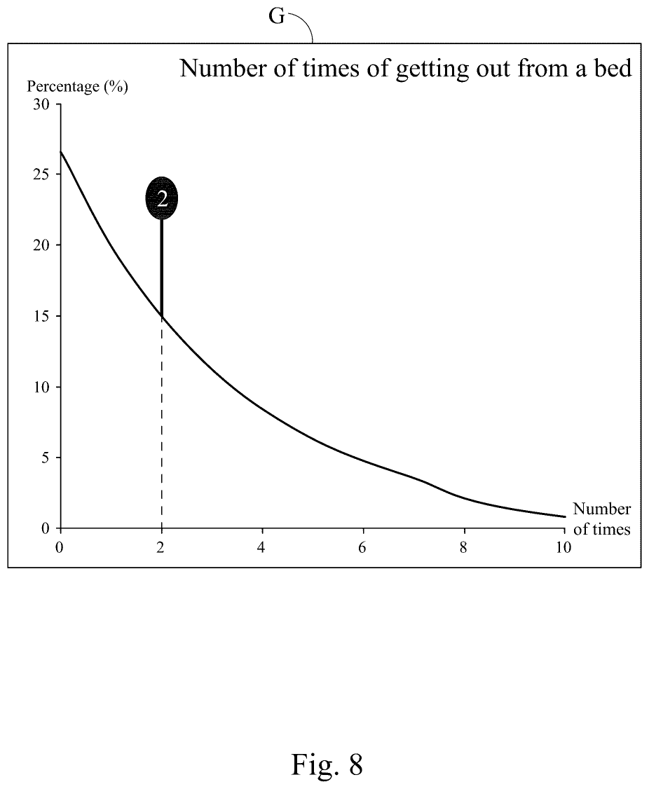

FIG. 6 is a schematic view of a sleeping index graphics G according to the 3rd example of the 1st embodiment of FIG. 2. FIG. 7 is a schematic view of a sleeping index graphics G according to the 4th example of the 1st embodiment of FIG. 2. FIG. 8 is a schematic view of a sleeping index graphics G according to the 5th example of the 1st embodiment of FIG. 2.

According to FIG. 2, FIG. 6, FIG. 7 and FIG. 8, in the 3rd example of the 1st embodiment, the sleeping score conversion unit 114 converts the sleeping analysis results into the sleeping index graphics G, the sleeping index graphics is donut chart. As shown in FIG. 6, the sleeping positions include supine position, left lateral position, right lateral position, prone position and other sleeping positions, each sleeping position has a corresponding ratio during the user's sleeping and a corresponding smoothness of sleeping breathing. The donut chart displays sleeping position and sleeping breathing at the same time, which helps users understand the correlation between sleeping position and sleeping breathing, and helps users find the best sleeping position to improve sleeping quality.

In the 4th example of the 1st embodiment, the sleeping score conversion unit 114 converts the sleeping analysis results corresponding to number of times of rolling over into the sleeping index graphics G, the sleeping index graphics G is curve chart. As shown in FIG. 7, the x-axis of the curve chart is the number of times of rolling over, and the y-axis of the curve chart is the proportion of sleeping analysis results in the data group, different number of times of rolling over has corresponding percentages. The curve chart displays the number of times of rolling over, which can help users understand the suitability of bedding and help users improve sleeping quality by selecting bedding.

In the 5th example of the 1st embodiment, the sleeping score conversion unit 114 converts the sleeping analysis results corresponding to number of times of getting out from the bed into the sleeping index graphics G, the sleeping index graphics G is curve chart. As shown in FIG. 8, the x-axis of the curve chart is the number of times of getting out from the bed, and the y-axis of the curve chart is the proportion of sleeping analysis results in the data group, different number of times of getting out from the bed has corresponding percentages. The curve chart displays the number of times of getting out from the bed, which can help users understand the degree of frequent urination.

Hence, the analysis module 110 can convert complex sleeping data into simple sleeping analysis result through the comparison and analysis functions of the analysis module so as to improve analysis efficiency greatly. The analysis module 110 provides users with easy-to-understand sleeping quality analysis through a variety of display functions. Therefore, it is favorable for helping users improve their understanding of their own sleeping quality. By displaying the radar chart, the distribution of the pros and cons of each sleeping index grade G is presented concisely so as to help users evaluate and improve the sleeping analysis result.

### 2nd Embodiment

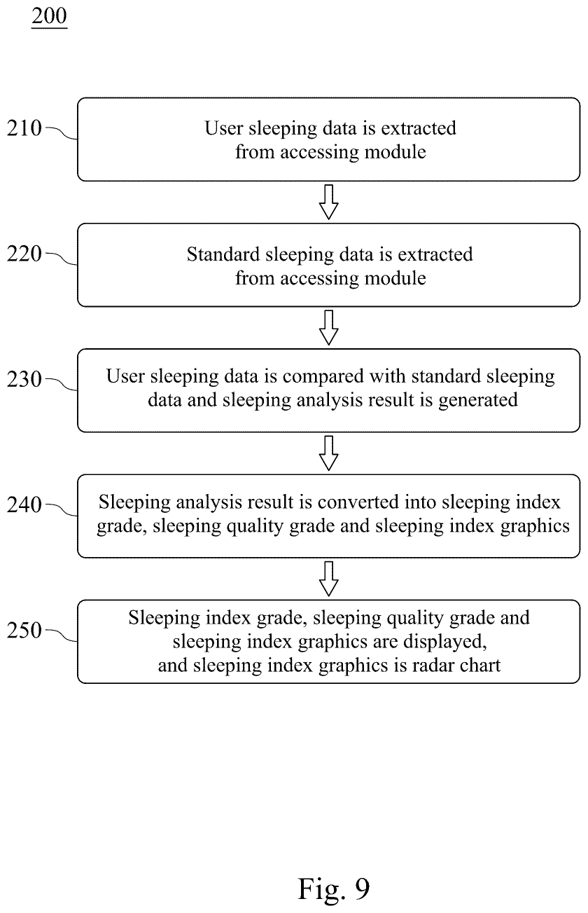

FIG. 9 is a flow chart of an analysis method 200 according to the 2nd embodiment of the present disclosure. According to FIG. 9, the analysis method 200 includes steps 210, 220, 230, 240, 250. In the step 210, the analysis module is driven to extract the user sleeping data from the accessing module. In the step 220, the analysis module is driven to extract the standard sleeping data from the accessing module corresponding to the user sleeping data. In the step 230, the analysis module is drive to compare the user sleeping data with the standard sleeping data and generate sleeping analysis result. In the step 240, the analysis module is driven to convert the sleeping analysis result into the sleeping index grade, the sleeping quality grade and the sleeping index graphics. In the step 250, the analysis module is driven to display the sleeping index grade, the sleeping quality grade and the sleeping index graphics, and the sleeping index graphics is a radar chart.

In detail, the aforementioned analysis system 100 of the 1st embodiment in FIG. 1 is configured to implement the analysis method 200 of the 2nd embodiment to quantify sleeping indicators and present user's sleeping status and present the sleeping quality. It should be noted that, the analysis method 200 of the present disclosure is not limited to implementation through the analysis system 100 of the present disclosure. Each component in the analysis system 100 can be arbitrarily integrated into various combinations to perform the functions of the analysis method 200.

Hence, it is favorable for greatly improving analysis efficiency by the method that can quantify various sleeping indicators to convert complex sleeping data into simple sleeping analysis result. By providing a simple and easy-to-understand sleeping quality analysis method to users, it is favorable for helping users improve their understanding of their own sleeping quality. By displaying the radar chart, the distribution of the pros and cons of each sleeping index grade is presented concisely so as to help users evaluate and improve the sleeping analysis result.

### 3rd Embodiment

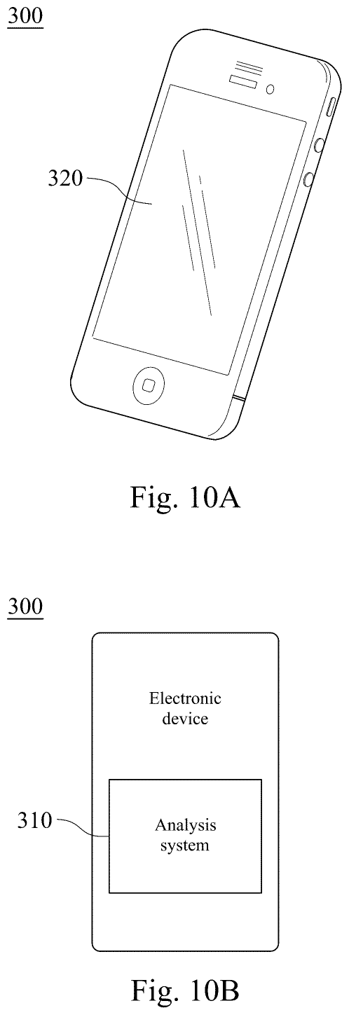

FIG. 10A is a schematic view of an electronic device 300 according to the 3rd embodiment of the present disclosure. FIG. 10B is a block diagram of the electronic device 300 according to the 3rd embodiment of FIG. 10A. As shown in FIG. 10A and FIG. 10B, the electronic device 300 of the 3rd embodiment is a smart phone. The electronic device 300 is signally connected to a sleeping monitoring instrument (not shown) to obtain various physiological activity indices (such as heartbeat, body temperature, blood pressure, sleeping time, sleeping position and respiratory rate) for analysis, and simply present the user's sleeping quality, providing users with an easy-to-understand sleeping quality analysis. Therefore, it is favorable for helping users improve their understanding of their own sleeping quality. The electronic device 300 includes an analysis system 310 and a user interface 320. In 3rd embodiment, the analysis system 310 can be the same as or similar to the analysis system 100 of the 1st embodiment and will not be described again herein. The user interface 320 is a touch screen of a smartphone, the contents displayed by the analysis system 310 on the user interface 320 can be the same as or similar to the contents displayed by the user interface UI of the 1st embodiment and will not be described again herein.

The foregoing description, for purpose of explanation, has been described with reference to specific embodiments. It is to be noted that Tables show different data of the different embodiments; however, the data of the different embodiments are obtained from experiments. The embodiments were chosen and described in order to best explain the principles of the disclosure and its practical applications, to thereby enable others skilled in the art to best utilize the disclosure and various embodiments with various modifications as are suited to the particular use contemplated. The embodiments depicted above and the appended drawings are exemplary and are not intended to be exhaustive or to limit the scope of the present disclosure to the precise forms disclosed. Many modifications and variations are possible in view of the above teachings.

What is claimed is:

1. An analysis module, comprising:
a user sleeping data extracting unit configured to extract a user sleeping data from an accessing module;
a standard sleeping data extracting unit configured to extract a standard sleeping data from the accessing module corresponding to the user sleeping data;
a sleeping data analysis unit configured to compare the user sleeping data with the standard sleeping data and generate at least one sleeping analysis result;
a sleeping score conversion unit configured to convert the at least one sleeping analysis result into at least one sleeping index grade, a sleeping quality grade and a sleeping index graphics; and
a sleeping score displaying unit configured to display the at least one sleeping index grade, the sleeping quality grade and the sleeping index graphics, and the sleeping index graphics is a radar chart.

2. The analysis module of claim 1, wherein the at least one sleeping analysis result comprises at least one of slow wave power, breathing power, falling asleep power, sustainability and sleeping length power.

3. The analysis module of claim 1, wherein the sleeping index grade is arranged around the sleeping index graphics.

4. The analysis module of claim 3, wherein the sleeping score displaying unit displays an indicator numeric score.

5. The analysis module of claim 4, wherein the sleeping score displaying unit displays a full score of indicator numeric, and the full score of indicator numeric overlaps the sleeping index graphics.

6. The analysis module of claim 1, wherein the sleeping score displaying unit displays a quality letter grade.

7. The analysis module of claim 1, wherein the sleeping score displaying unit displays a full score of quality numeric.

8. The analysis module of claim 1, wherein the sleeping score displaying unit displays an indicator percentage.

9. The analysis module of claim 1, wherein the sleeping score displaying unit displays a quality percentage.

10. The analysis module of claim 1, further comprising a sleeping interval displaying unit.

11. The analysis module of claim 1, further comprising a sleeping position displaying unit.

12. The analysis module of claim 1, further comprising a sleeping advice displaying unit.

13. An analysis system, comprising:
the analysis module of claim 1; and
the accessing module connected to the analysis module.

14. An electronic device, comprising:
the analysis system of claim 13.

15. An analysis module, comprising:
a user sleeping data extracting unit configured to extract a user sleeping data from an accessing module;
a standard sleeping data extracting unit configured to extract a standard sleeping data from the accessing module corresponding to the user sleeping data;
a sleeping data analysis unit configured to compare the user sleeping data with the standard sleeping data and generate at least three sleeping analysis results;
a sleeping score conversion unit configured to convert the at least three sleeping analysis results into at least three sleeping index grades, at least one sleeping quality grade and a sleeping index graphics, and the at least three sleeping analysis results comprise at least one of slow wave power, breathing power, falling asleep power, sustainability and sleeping length power; and
a sleeping score displaying unit configured to display the at least three sleeping index grades, the sleeping quality grade and the sleeping index graphics.

16. The analysis module of claim 15, wherein the at least three sleeping index grades overlap the sleeping index graphics.

17. The analysis module of claim 16, wherein the sleeping score displaying unit displays an indicator numeric score.

18. The analysis module of claim 17, wherein the sleeping score displaying unit displays an indicator percentage.

19. The analysis module of claim 18, wherein the sleeping index graphics is a radar chart.

20. The analysis module of claim 15, wherein the sleeping score displaying unit displays a full score of quality numeric.

21. The analysis module of claim 20, wherein the sleeping score displaying unit displays a quality percentage.

22. The analysis module of claim 21, wherein the sleeping score displaying unit displays a quality letter grade.

23. The analysis module of claim 22, further comprising a sleeping interval displaying unit.

24. The analysis module of claim 23, further comprising a sleeping position displaying unit.

25. The analysis module of claim 24, wherein the sleeping index graphics is a donut chart, the donut chart is transformed by at least two sleeping analysis results, and the at least two sleeping analysis results comprise sleeping position and sleeping breathing.

26. The analysis module of claim 25, wherein the sleeping index graphics is a curve chart, the curve chart is transformed by at least one sleeping analysis result, and the at least one sleeping analysis result comprises a number of times of rolling over and a number of times of getting out from a bed.

27. The analysis module of claim 26, further comprising a sleeping advice displaying unit.

28. An analysis system, comprising:
the analysis module of claim 15; and
the accessing module connected to the analysis module.

29. An electronic device, comprising:
the analysis system of claim 28.

30. An analysis method, comprising the steps of:
driving an analysis module to extract a user sleeping data from an accessing module;
driving the analysis module to extract a standard sleeping data from the accessing module corresponding to the user sleeping data;
driving the analysis module to compare the user sleeping data with the standard sleeping data and generate at least one sleeping analysis result;
driving the analysis module to convert the at least one sleeping analysis result into at least one sleeping index grade, a sleeping quality grade and a sleeping index graphics;
driving the analysis module to display the at least one sleeping index grade, the sleeping quality grade and the sleeping index graphics, and the sleeping index graphics is a radar chart.
